(19) United States
(12) Patent Application Publication
(10) Pub. No.: US 2017/0056252 A1
(43) Pub. Date: Mar. 2, 2017

(54) PRECUT KINESIOLOGY TAPE FOR WRIST SUPPORT

(71) Applicant: SPIDERTECH INC., Toronto (CA)

(72) Inventor: Ray Arbesman, Toronto (CA)

(73) Assignee: SPIDERTECH INC., Toronto (CA)

(21) Appl. No.: 15/243,077

(22) Filed: Aug. 22, 2016

Related U.S. Application Data
(60) Provisional application No. 62/212,042, filed on Aug. 31, 2015.

(51) Int. Cl. A61F 13/10; A61F 13/02; A61F 5/01

(52) U.S. Cl. CPC A61F 13/107; A61F 5/0118; A61F 13/025

(57) ABSTRACT

A precut kinesiology tape for wrist support includes a strip of woven material having a first face and an opposed second face, a length and a width that is shorter than the length, and a length stretch ratio along the length and a width stretch ratio along the width. The width stretch ratio is greater than the length stretch ratio. An adhesive is on the first face in a discontinuous pattern.

### CROSS-REFERENCES TO RELATED APPLICATIONS

This application claims the benefit of U.S. Provisional Patent application No. 62/212,042, filed on Aug. 31, 2015, which is incorporated herein by reference in its entirety.

### FIELD

The specification relates to precut kinesiology tapes. More particularly, the specification relates to precut kinesiology tapes for wrist support, and related kits and methods.

### BACKGROUND

U.S. Pat. No. 2011/0275969 (Quinn) purports to disclose a pre-cut strip of kinesiology tape. The pre-cut strip of kinesiology tape includes a fabric. The fabric includes a weave of fibers, where the fibers include an elastic fiber covered by a covering material. The fabric also includes a first end and a second end, where the second end is opposite the first end. The fabric further includes one or more rounded corners. The pre-cut strip of kinesiology tape also includes a longitudinal cut in the fabric. The longitudinal cut passes through at least a portion of the fabric and extends from the first end to a pre-determined distance from the second end. The pre-cut strip of kinesiology tape also includes adhesive on a first surface of the fabric, where the adhesive is configured to adhere the fabric to a human body.

### SUMMARY

The following summary is intended to introduce the reader to various aspects of the specification, but not to define or delimit any invention.

According to some aspects, a precut kinesiology tape for wrist support is disclosed. The precut kinesiology tape comprises a strip of woven material having a first face and an opposed second face, a length and a width, wherein the width is shorter than the length, and a length stretch ratio along the length and a width stretch ratio along the width. The width stretch ratio is greater than the length stretch ratio. An adhesive is on the first face in a discontinuous pattern.

The strip may have a high width stretch ratio, and a low length stretch ratio. The strip may be at least twice as stretchable in a widthwise direction than in a lengthwise direction. The width stretch ratio may be at least 1.4, or at least 1.6. The length stretch ratio may be at most 1.3, or at most 1.2.

The length may be between about 130 mm and 270 mm, and the width may be between about 45 mm and 100 mm. The length may be between about 160 mm and about 240 mm, and the width may be between about 55 mm and about 90 mm. The length may be at least about 2 times the width, or at least about 2.5 times the width, or at least about 2.75 times the width.

The material may comprise woven cotton.

The tape may extend lengthwise between a first end and a second end. The first end may comprise a first alignment feature, and the second end may comprise a second alignment feature for aligning with the first alignment feature.

The adhesive may be provided in bands, and the bands may extend generally widthwise across the tape. The bands may be provided in a wave pattern.

According to some aspects, a method of supporting a wrist is disclosed. The method comprises a) positioning a piece of kinesiology tape adjacent the wrist so that a greater stretch axis of the tape is generally aligned along the wrist and a lesser stretch axis of the tape extends generally transverse to the wrist; b) wrapping the piece of kinesiology tape at least partially around the wrist, wherein during step b) the kinesiology tape is at most stretched minimally along the low stretch axis; and c) adhering the piece of kinesiology tape to the wrist.

The kinesiology tape may have an unstretched length, and during step b), the kinesiology tape may be stretched to a stretched length that is at most 1.3 times the unstretched length, or at most 1.2 times the unstretched length.

The piece of kinesiology tape may have an unstretched width, and may be stretchable to a stretched width that is at least 1.4 times the unstretched width. During step b), the piece of kinesiology tape may be stretched at most minimally along the high stretch axis.

A release liner may be provided on the piece of kinesiology tape. The method may further comprise, prior to step a), removing a central portion of the release liner from the piece of kinesiology tape.

Steps b) and c) may comprise: i) adhering a central portion of the piece of kinesiology tape to the wrist; ii) removing end portions of the release liner from the end portions of the piece of kinesiology tape; iii) wrapping the end portions of the piece of kinesiology tape at least partially around the wrist; and iv) adhering the end portions of the piece of kinesiology tape to the wrist.

According to some aspects, a kit for wrist support is disclosed. The kit comprises a precut kinesiology tape. The tape comprises a strip of woven material having a first face and an opposed second face, a length and a width, wherein the width is shorter than the length, and a length stretch ratio along the length and a width stretch ratio along the width. The width stretch ratio is greater than the length stretch ratio. An adhesive is on the first face in a discontinuous pattern. The kit further comprises a release liner on the first face. The release liner comprises a first line of weakness and a second line of weakness spaced apart from the first line of weakness in a lengthwise direction. The first line of weakness and the second line of weakness extend a widthwise direction.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example precut kinesiology tape;

FIG. 2 is an enlarged view of the region shown in circle 2 in FIG. 1;

FIG. 3 is a top view of the tape of FIG. 1;

FIG. 4 is a bottom view of the tape of FIG. 1;

FIG. 5A is a top view of the tape of FIG. 1, in an unstretched configuration;

FIG. 5B is a top view of the tape of FIG. 5A, in a stretched configuration;

FIG. 6 is a side view of a kit including the tape of FIG. 1 on a release liner;

FIG. 7 is a bottom view of the kit of FIG. 6;

FIG. 8 is a bottom view of the kit of FIG. 6, with a central portion of the release liner removed from the tape;

FIG. 9 is a schematic top view of a forearm and wrist, with the tape of FIG. 8 positioned on the wrist;

FIG. 10 is a schematic bottom view of the forearm and wrist of FIG. 9, with the tape of FIG. 9 wrapped around the wrist;

FIG. 11 is a perspective view of an example roll of kinesiology tape, showing how the tape of FIG. 1 may be cut therefrom in order to fabricate the precut tape; and

FIG. 12 is a top view of an alternative example tape.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Disclosed herein is a precut kinesiology tape, and related kits and methods, which may be used for wrist support. For example, the precut kinesiology tape may be used to treat, prevent, or inhibit wrist pain, or to treat, prevent, or inhibit wrist injuries. Such wrist pain and injuries may be associated with, for example, carpal tunnel syndrome, sprains, tendonitis, arthritis, and/or gout.

In some examples, the precut kinesiology tape may be wrapped at least partially around a wrist, to support the wrist. As will be described in further detail below, the precut kinesiology tape may configured such that it is highly stretchable along its width, but minimally stretchable or non-stretchable along its length. In use, when wrapped around a wrist, the width of the tape may extend along and be generally aligned with the wrist (i.e. generally parallel to the forearm), and the length of the tape may be generally transverse to the wrist (i.e. generally perpendicular to the forearm). Because the tape is minimally stretchable or non-stretchable along the length, in use, over-compression of the wrist may be avoided, inhibited, or prevented. Furthermore, because the tape is highly stretchable along its width, in use, interference with wrist movement may be avoided, inhibited, or prevented, and comfort may be enhanced.

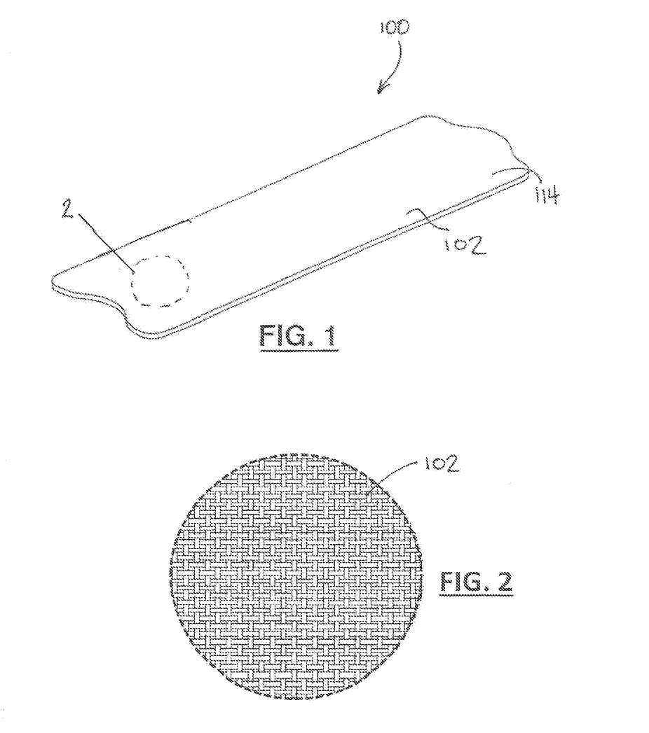

Referring now to FIG. 1, an example precut kinesiology tape 100 is shown. The tape 100 includes a strip 102 of woven material. The woven nature of the strip 102 is shown schematically in FIG. 2. The woven material may be or may include, for example, woven cotton, or any other material suitable for use in kinesiology tape.

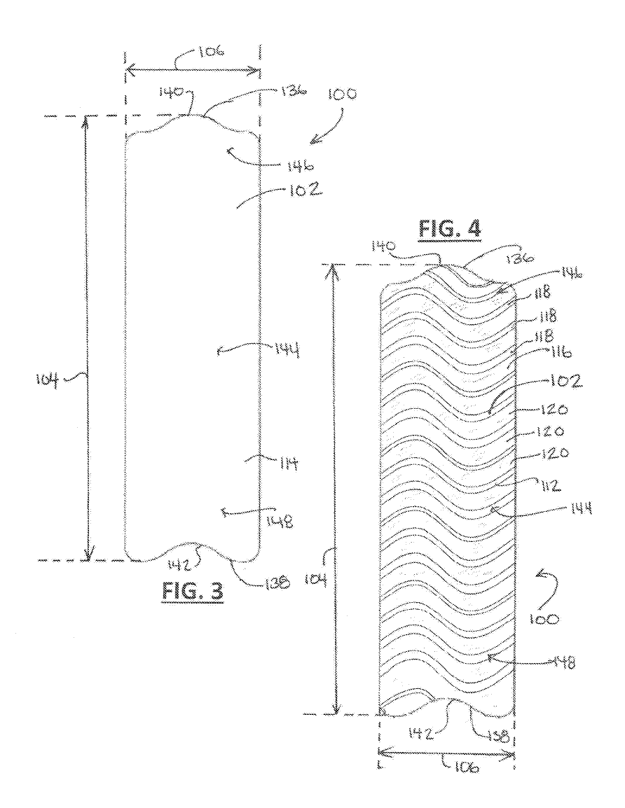

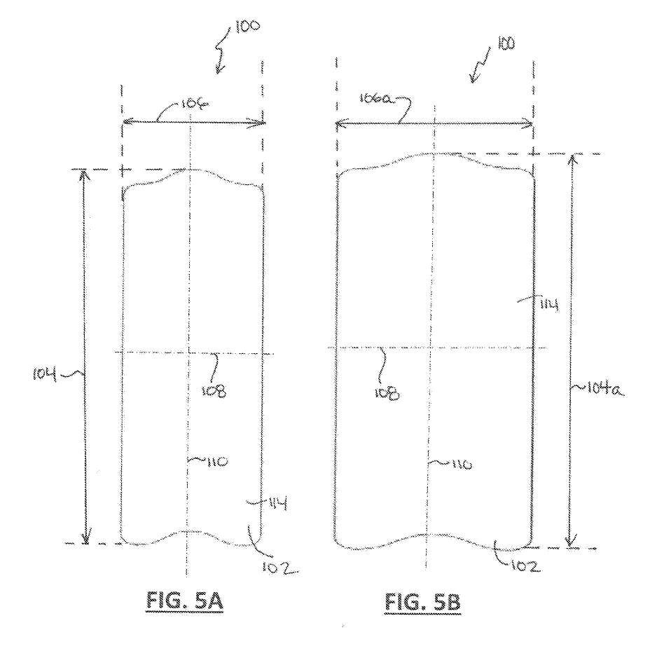

Referring to FIGS. 3 and 4, the strip 102 has a length 104 and a width 106 (wherein length 104 and width 106 refer to the dimensions when the tape 100 is in an unstretched or natural configuration). The width 106 is shorter than the length 104. For example, the length 104 may be at least about 2 times the width 106, or at least about 2.5 times the width 106, or at least about 2.75 times the width 106. In some examples, the length 104 may be between about 130 mm and 270 mm, and the width 106 may be between about 45 mm and 100 mm. In some examples, the length 104 may be between about 160 mm and about 240 mm, and the width 106 may be between about 55 mm and about 90 mm. In one particular example, the length 104 is about 178 mm, and the width 106 is about 60 mm. In another particular example, the length 104 is about 203 mm, and the width 106 is about 70 mm. In another example, the length 104 is about 216 mm, and the width 106 is about 80 mm.

The strip 102 has a stretch ratio along its length 104 (also referred to as a length stretch ratio), and a stretch ratio along its width 106 (also referred to as a width stretch ratio). As used herein, the term ‘stretch ratio’ refers to a dimension of the strip 102 when stretched elastically to its maximum extent, divided by a dimension of the strip 102 when unstretched. For example, referring to FIG. 5A, the tape 100 is shown in an unstretched or natural configuration. Referring to FIG. 5B, the tape 100 is shown in a stretched configuration, wherein the strip 102 is stretched elastically to its maximum extent in both a widthwise and lengthwise direction (i.e. along transverse axis 108 and longitudinal axis 110). The width 106a of the strip 102 when the strip 102 is stretched elastically to its maximum extent along transverse axis 108, divided by its width 106 when unstretched, is its width stretch ratio. The length 104a of the tape when the tape is stretched elastically to its maximum extent along longitudinal axis 110, divided by its length 104 when unstretched, is its length stretch ratio.

In the example shown, the width stretch ratio is greater than the length stretch ratio. For example, the strip 102 may be at least twice as stretchable in a widthwise direction than in a lengthwise direction. For further example, the width stretch ratio may be at least 1.4 or about 1.4, and the length stretch ratio may be at most 1.3 or about 1.3. For further example, the width stretch ratio may be at least 1.6 or about 1.6, and the length stretch ratio may be at most 1.2 or about 1.2. For further example, the length stretch ratio may be between 1.4 and 2.0, and the width stretch ratio may be between 1.0 and 1.3.

In this document, a stretch ratio of 1.4 or greater may be referred to as a ‘high’ stretch ratio, and a stretch ratio of 1.3 or less (including a stretch ratio of 1) may be referred to as a ‘low’ stretch ratio. A material with a stretch ratio of 1 may also be referred to as ‘non-stretchable’. Furthermore, the transverse axis 108, along which the strip 102 has a greater stretch ratio, may be referred to as a ‘greater stretch axis’. The longitudinal axis 110, along which the strip 102 has a lower stretch ratio, may be referred to as a ‘lesser stretch axis’.

Referring back to FIGS. 3 and 4, the strip 102 has a first face 112 (shown in FIG. 4), and an opposed second face 114 (shown in FIG. 3). In use, the first face 112 faces the skin of the user, and the second face 114 faces away from the skin of the user.

An adhesive 116 is on the first face 112. Referring still to FIG. 4, in the example shown, the adhesive 116 is on the first face 112 in a discontinuous pattern. That is, the first face 112 includes a set of adhesive-covered portions (i.e. portions that are covered by the adhesive 116), and a set of adhesive-free portions 118 (i.e. portions that are free of adhesive 116, so that the first face is exposed). The adhesive-covered portions and adhesive-free portions 118 are provided in an alternating pattern, so that the adhesive-covered portions are spaced apart from each other, and the adhesive-free portions 118 are spaced apart from each other.

Referring still to FIG. 4, in the example shown, the adhesive 116 is provided on the adhesive covered portions in bands 120. The bands 120 extend non-linearly, and in the example shown are generally wave-shaped. The wave-shaped bands 120 extend generally widthwise across the tape, along the transverse axis 108 (i.e. along the greater stretch axis 108, shown in FIGS. 5A and 5B).

This discontinuous pattern of the adhesive 110 may allow for the tape 100 to accommodate movement, even when adhered to skin.

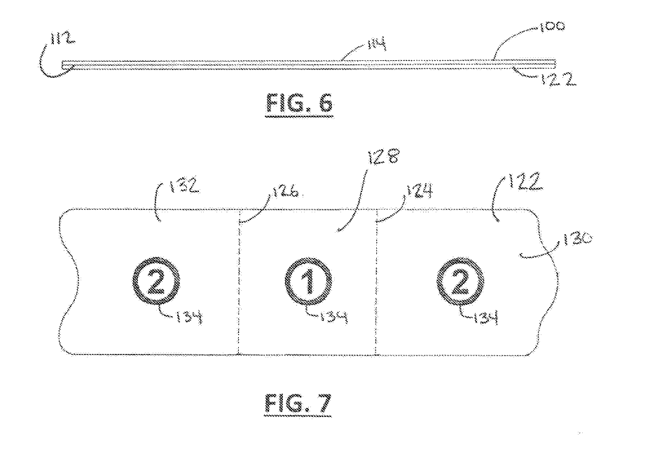

Referring to FIGS. 6 and 7, in the example shown, the tape 100 is provided in a kit with a release liner 122. The release liner 122 is adhered to the first face 112. The release liner 122 may in some examples include one or more lines of weakness, so that it may be severed along the lines of weakness and the portions thereof removed stepwise, in order to facilitate stepwise application of the tape 100. In the example shown, the release liner includes a first line of weakness 124 and a second line of weakness 126. In the example shown, each line of weakness 124, 126 is a perforated line. In alternative examples, the lines of weakness may be creases or score-lines.

Referring still to FIGS. 6 and 7, in the example shown, the first 124 and second 126 lines of weakness are spaced apart in a lengthwise direction, along the longitudinal axis 110 (i.e. lesser stretch axis 110, shown in FIGS. 5A and 5B), and extend in a widthwise direction, along the transverse axis 108 (i.e. greater stretch axis 108, shown in FIGS. 5A and 5B). The first 124 and second 126 lines of weakness demarcate a central portion 128 of the release liner 122, and first 130 and second 132 end portions of the release liner 122.

Referring still to FIG. 7, in the example shown, the release liner 122 further includes markings 134 to assist the user in the application of the tape 100. The markings as shown are numerical markings, indicating an order in which the central portion 128 and end portions 130, 132 of the release liner 122 may be removed, in order to facilitate stepwise application of the tape 100. In other examples, the markings may be alphabetical markings, or pictorial markings. In other examples, markings may be provided elsewhere, for example on the tape or on a separate instruction sheet.

Referring back to FIGS. 3 and 4, in the example shown, the strip 102 extends lengthwise between a first end 136 and an opposed second end 138. The first end 136 and second end 138 include first 140 and second 142 alignment features, respectively. In use, as will be described below, the first alignment feature 140 may be aligned with the second alignment feature 142, in order to facilitate generally straight application of the tape 100 around the wrist. In the example shown, the first alignment feature 140 is a protrusion, and the second alignment feature 142 is an indentation shaped to match the protrusion.

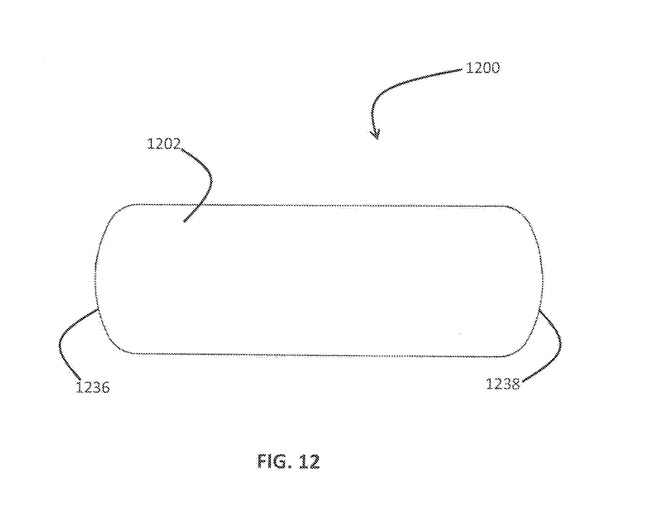

Referring now to FIG. 12, an alternative example tape 1200 is shown, in which like numerals are used to refer to like features as in FIGS. 1 to 7, with the reference numerals incremented by 11. In the tape 1200, the first end 1236 and second end 1238 of the strip 1202 are rounded, and do not include any alignment features such as protrusions or indentations.

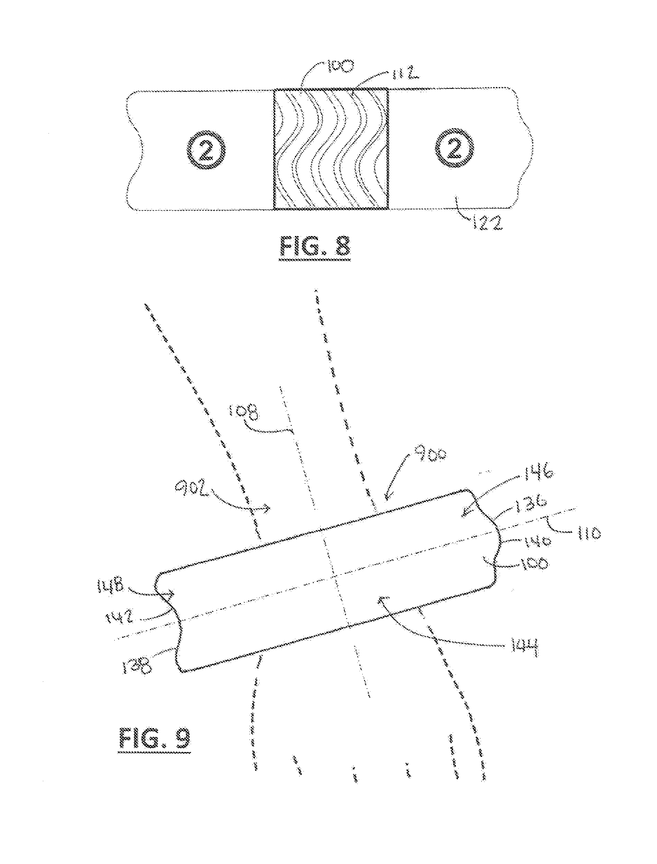

Referring now to FIGS. 8 to 10, a method of supporting a wrist will be described. The method will be described with respect to precut kinesiology tape 100; however, the method may be carried out with other tapes, and tape 100 may be used according to other methods.

Referring to FIG. 8, the release liner 122 may be torn along the first 124 and second 126 lines of weakness (shown in FIG. 7), and the central portion 128 (shown in FIG. 7) of the release liner 122 may be removed from the tape 100.

Referring to FIG. 9, the tape 100 may be positioned adjacent a wrist 900 to be supported, on the dorsal side 902 of the wrist 900. The tape 100 may be positioned so that the first face 112 (shown in FIG. 8) faces the wrist 900, and so that the transverse axis 108 (i.e. greater stretch axis 108) is generally aligned along the wrist (i.e. generally parallel to the forearm), and the longitudinal axis 110 (i.e. lesser stretch axis 110) extends generally transverse to the wrist (i.e. generally perpendicular to the forearm). A central portion 144 of the tape 100 (i.e. the portion exposed by the removal of the central portion 128 of the release liner 122) may then be adhered to the wrist 900. For example, the central portion 144 of the tape 100 may be pressed against the wrist 900 and optionally rubbed.

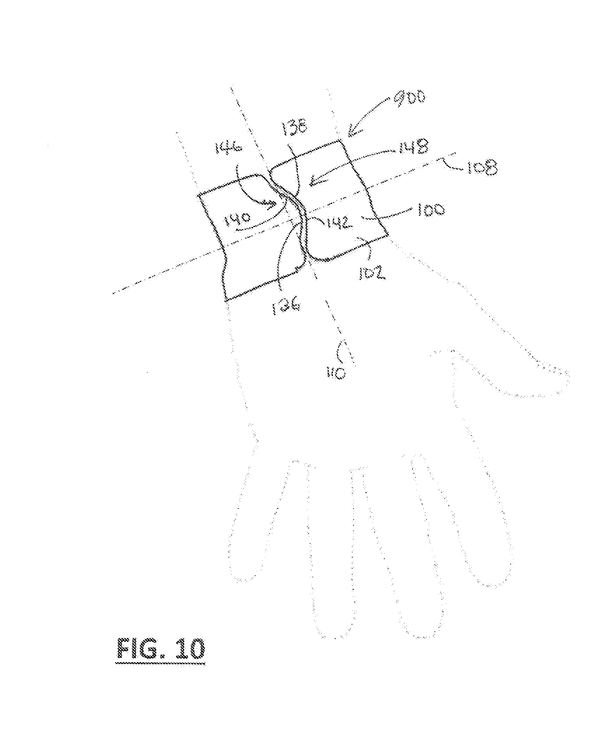

Referring to FIG. 10, the tape 100 may then be wrapped at least partially around the wrist 900 and adhered to the wrist 900. For example, the end portions 130, 132 of the release liner (shown in FIG. 7) may be removed from the tape 100, and the end portions 146, 148 of the tape (i.e. the portions exposed by the removal of the end portions 130, 132 of the release liner) may be wrapped at least partially around the wrist 900 and adhered to the wrist 900.

In some examples, as shown in FIG. 10, the tape 100 may be wrapped partially around the wrist 900, so that the first 136 and second 138 ends of the strip 102 are adjacent but spaced apart. Alternatively, the tape 100 may be wrapped fully around the wrist 900, so that the first 136 and second 138 ends of the strip 102 are adjacent and touching. Alternatively, the tape 100 may be wrapped fully around the wrist 900, so that the first 136 and second 138 ends of the strip 102 are adjacent and overlapping.

The alignment features 140 and 142 may facilitate the wrapping of the tape 100 in a straight fashion, and the tape 100 may be wrapped so that the protrusion of the first alignment feature 140 is received in or pointing towards the indentation of the second alignment feature 142.

To adhere the tape 100 to the wrist 900, the tape may be pressed against the wrist 900, and optionally rubbed.

Because the strip 102 has a low stretch ratio along the longitudinal axis 110, the strip 102 may remain minimally stretched or non-stretched (i.e. at most minimally stretched) as it is wrapped around the wrist 900. For example, the strip 102 may be stretched to a stretched length that is at most 1.3 times its unstretched length, or at most 1.2 times its unstretched length. This may avoid, inhibit, or prevent over-compression of the wrist 900 by the tape 100. This in turn may enhance comfort.

Furthermore, even though the strip 102 has a high stretch ratio along the transverse axis 108, it may be applied in a non-stretched or minimally stretched configuration along the transverse axis 108.

Because the strip 102 has a high stretch ratio along the transverse axis 108, the tape 100 may support the wrist 900 while avoiding, inhibiting, or preventing interference with wrist movement. For example, the tape 100 may support the wrist 900 while still allowing for flexion and extension of the wrist 900, and radial and ulnar deviation of the wrist 900. Furthermore, comfort may be enhanced.

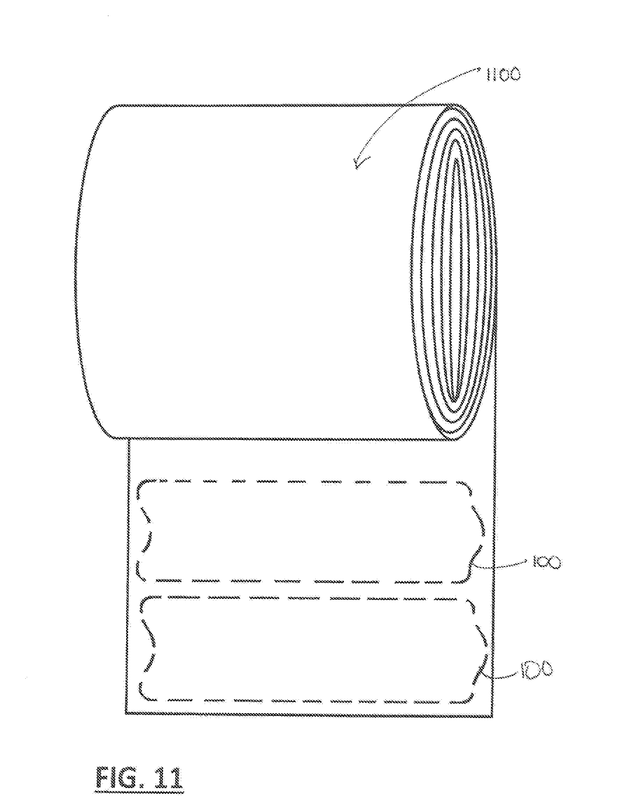

Referring to FIG. 11, a schematic diagram is shown illustrating how the tape 100 may be cut from a commercially available roll 1100 of kinesiology tape, in order to provide precut tapes. The roll 1100 of kinesiology tape may have a high stretch ratio along its length and a low stretch ratio across its width. The tape 100 may be cut so that its width extends transversely across the roll 1100, to provide the tape 100 with a high stretch ratio in its widthwise direction. The shapes labelled as tape 100 in FIG. 11 are intended to illustrate the orientation of the tape 100 with respect to the roll 1100 as the tapes 100 are cut from the roll.

While the above description provides examples of one or more processes or apparatuses, it will be appreciated that other processes or apparatuses may be within the scope of the accompanying claims.

1. A precut kinesiology tape for wrist support, comprising:
a) a strip of woven material having i) a first face and an opposed second face, ii) a length and a width, wherein the width is shorter than the length; and iii) a length stretch ratio along the length and a width stretch ratio along the width, wherein the width stretch ratio is greater than the length stretch ratio; and
b) an adhesive on the first face in a discontinuous pattern.

2. The precut kinesiology tape of claim 1, wherein the strip is at least twice as stretchable in a widthwise direction than in a lengthwise direction.

3. The precut kinesiology tape of claim 1, wherein the width stretch ratio is at least 1.4.

4. The precut kinesiology tape of claim 1, wherein the width stretch ratio is at least 1.6.

5. The precut kinesiology tape of claim 1, wherein the length stretch ratio is at most 1.3.

6. The precut kinesiology tape of claim 1, wherein the length stretch ratio is at most 1.2.

7. The precut kinesiology tape of claim 1, wherein the strip has a high width stretch ratio, and a low length stretch ratio.

8. The precut kinesiology tape of claim 1, wherein the length is between about 130 mm and 270 mm, and the width is between about 45 mm and 100 mm.

9. The precut kinesiology tape of claim 1, wherein the length is between about 160 mm and about 240 mm, and the width is between about 55 mm and about 90 mm.

10. The precut kinesiology tape of claim 1, wherein the length is at least about 2 times the width.

11. The precut kinesiology tape of claim 1, wherein the length is at least about 2.5 times the width.

12. The precut kinesiology tape of claim 1, wherein the length is at least about 2.75 times the width.

13. The precut kinesiology tape of claim 1, wherein the material comprises woven cotton.

14. The precut kinesiology tape of claim 1, wherein the tape extends lengthwise between a first end and a second end, the first end comprises a first alignment feature, and the second end comprises a second alignment feature for aligning with the first alignment feature.

15. The precut kinesiology tape of claim 1, wherein the adhesive is provided in bands, and the bands extend generally widthwise across the tape.

16. The precut kinesiology tape of claim 15, wherein the bands are provided in a wave pattern.

17. A method of supporting a wrist, comprising:
a) positioning a piece of kinesiology tape adjacent the wrist so that a greater stretch axis of the tape is generally aligned along the wrist and a lesser stretch axis of the tape extends generally transverse to the wrist;
b) wrapping the piece of kinesiology tape at least partially around the wrist, wherein during step b) the kinesiology tape is at most stretched minimally along the low stretch axis; and
c) adhering the piece of kinesiology tape to the wrist.

18. The method of claim 17, wherein the kinesiology tape has an unstretched length, and during step b), the kinesiology tape is stretched to a stretched length that is at most 1.3 times the unstretched length.

19. The method of claim 17, wherein during step b), the kinesiology tape is stretched to a stretched length that is at most 1.2 times the length.

20. The method of claim 17, wherein the piece of kinesiology tape has an unstretched width, and is stretchable to a stretched width that is at least 1.4 times the unstretched width.

21. The method of claim 17, wherein during step b), the piece of kinesiology tape is stretched at most minimally along the high stretch axis.

22. The method of claim 17, wherein a release liner is provided on the kinesiology tape, and the method further comprises, prior to step a) removing a central portion of the release liner from the kinesiology tape.

23. The method of claim 22, wherein steps b) and c) comprise:
i) adhering a central portion of the kinesiology tape to the wrist;
ii) removing end portions of the release liner from the end portions of the kinesiology tape;
iii) wrapping end portions of the piece of kinesiology tape at least partially around the wrist; and
iv) adhering the end portions of the kinesiology tape to the wrist.

24. A kit for wrist support, comprising:
a) a tape comprising i) a strip of woven material having a first face and an opposed second face, a length and a width, wherein the width is shorter than the length, and a length stretch ratio along the length and a width stretch ratio along the width, wherein the width stretch ratio is greater than the length stretch ratio, and ii) an adhesive on the first face in a discontinuous pattern; and
b) a release liner on the first face, the release liner comprising a first line of weakness and a second line of weakness spaced apart from the first line of weakness in a lengthwise direction, the first line of weakness and the second line of weakness extending in a widthwise direction.
